(19) United States
(12) Patent Application Publication
(10) Pub. No.: US 2022/0065875 A1
(43) Pub. Date: Mar. 3, 2022

(54) BIOMARKER COMPOSITION FOR DIAGNOSIS OF DEGENERATIVE BRAIN DISEASE

(71) Applicant: AJOU UNIVERSITY INDUSTRY-ACADEMIC COOPERATION FOUNDATION, Suwon-si (KR)

(72) Inventor: Sun Ah PARK, Suwon-si (KR)

(73) Assignee: AJOU UNIVERSITY INDUSTRY-ACADEMIC COOPERATION FOUNDATION, Suwon-si (KR)

(21) Appl. No.: 17/416,029

(22) PCT Filed: Feb. 7, 2020
(86) PCT No.: PCT/KR2020/001736

§ 371 Date: Jun. 18, 2021

(30) Foreign Application Priority Data
Feb. 8, 2019 (KR) 10-2019-0014992
Dec. 30, 2019 (KR) 10-2019-0178182

(51) Int. Cl. G01N 33/68; G01N 33/50

(52) U.S. Cl. CPC G01N 33/6896; G01N 2800/28; G01N 33/5008

(57) ABSTRACT

A biomarker composition for diagnosing degenerative brain diseases comprises TB (TRAF-6 binding) domain in SQSTM1/p62 protein. The TB (TRAF-6 binding) domain in the SQSTM1/p62 protein directly binds to the tau protein to remove the tau protein, and thus the exposure of the TB domain may be usefully utilized in diagnosing and/or treating degenerative brain diseases in degenerative brain diseases in which tauopathy is observed.

### CROSS REFERENCE TO RELATED APPLICATIONS

This application is a U.S. National Stage Application of International Application No. PCT/KR2020/001736, filed on Feb. 7, 2020, which claims the benefit under 35 USC 119(a) and 365(b) of Korean Patent Application No. 10-2019-0014992, filed on Feb. 8, 2019 and Korean Patent Application No. 10-2019-0178182, filed on Dec. 30, 2019, in the Korean Intellectual Property Office, the entire disclosure of which is incorporated herein by reference for all purposes.

### TECHNICAL FIELD

The present invention relates to a biomarker composition for diagnosing degenerative brain diseases.

### BACKGROUND ART

Degenerative brain disease refers to a disease that occurs in the brain among degenerative diseases that occur with age, and can be classified by considering the main symptoms and the affected brain region including representatively, Alzheimer's disease and frontotemporal dementia, Lewy body dementia, Parkinson's disease, Huntington's disease, and amyotrophic lateral sclerosis. Degenerative brain disease is known to be caused by neurodegeneration due to aging and by the death of neurons by the agglomeration of specific proteins for each disease due to genetic and environmental factors, however the exact pathologic mechanism has not yet been identified, and basic research is being actively conducted to clarify it.

Alzheimer's disease gradually develops due to decreased memory and progresses to progressive and extensive loss of cognitive functions such as loss of spatiotemporal ability, speech impairment and executive ability to show symptoms of dementia in which independent daily life cannot be performed. Although the pathological mechanism has not yet been accurately identified, it is reported to be due to the interaction between aging, genetic risk factors, and environmental factors.

Alzheimer's disease has been known to be mainly due to the accumulation of beta-amyloid and tau proteins, and research has been focused on removing amyloid plaques and neurofibrillary tangles formed by aggregation of these proteins. In recent years, as therapeutic agents targeting beta-amyloid fail in large-scale clinical drug research, tau protein has become more prominent as a major target for the development of therapeutic agents, and international attention is being focused. In addition, aggregation of tau protein is not limited to Alzheimer's disease, and is known to play a major role in the onset and progression of frontotemporal dementia, amyotrophic lateral sclerosis, and some Parkinson's disease, and thus therapeutic agents targeting tau protein can be expected to have a common therapeutic effect for several degenerative brain diseases.

It is known that tau protein is a material necessary to stabilize microtubules like rebar (reinforcement bar) connecting the railway and when tau protein is released from microtubules to accumulate excessively, the microtubules collapse and the network of normal neurons is disrupted. The most well-known mechanism for the accumulation of tau protein is the excessive adhesion of phosphorus to tau protein to form a heavy body due to hyperphosphorylation. The disease caused by the accumulation of these tau proteins and aggregation in nerve cells is collectively called tauopathy, and it has been pointed out as the cause of several degenerative brain diseases such as Alzheimer's disease, frontotemporal dementia, amyotrophic lateral sclerosis, some Parkinson's disease, progressive supranuclear palsy, corticobasal degeneration and Pick's disease.

Nevertheless, there are no clear therapies or therapeutic agents for diseases related to tauopathy. Therefore, in order to develop the ultimate therapeutic agent, it is necessary to sufficiently investigate the mechanism of toxicity and aggregation of tau protein, and to study a therapeutic agent capable of specifically inhibiting the mechanism.

### DISCLOSURE

### Technical Problem

An object of the present invention is to provide a biomarker composition for diagnosing degenerative brain diseases.

Another object of the present invention is to provide a kit for diagnosing degenerative brain diseases.

Another object of the present invention is to provide a method of providing information necessary for diagnosing degenerative brain diseases.

Another object of the present invention is to provide a method of screening a therapeutic agent for degenerative brain disease.

### Technical Solution

In order to achieve the above object, the present invention provides a biomarker composition for diagnosing degenerative brain diseases comprising TB (TRAF-6 binding) domain in SQSTM1/p62 protein.

The present invention also provides a kit for diagnosing degenerative brain diseases comprising an agent capable of measuring exposure level of TB (TRAF-6 binding) domain in SQSTM1/p62 protein.

In addition, the present invention provides a method of providing information necessary for diagnosing degenerative brain diseases comprising: (a) measuring an exposure level of TB (TRAF-6 binding) domain in tertiary structure of SQSTM1/p62 protein in a sample isolated from a patient; (b) comparing the exposure level of the TB domain with a control sample; and (c) determining as degenerative brain disease when the exposure level of the TB domain is lower than that of the control sample.

Furthermore, the present invention provides a method of screening a therapeutic agent for degenerative brain diseases comprising: (a) treating a candidate drug to a sample isolated from a patient; (b) measuring an exposure level of TB (TRAF-6 binding) domain in tertiary structure of SQSTM⁻I/p62 protein before and after treatment with the candidate drug; and (c) selecting a candidate drug that increases the exposure level of the TB domain compared to a control sample.

### Advantageous Effects

According to the present invention, the TB (TRAF-6 binding) domain in the SQSTM1/p62 protein directly binds to the tau protein to remove the tau protein, and the exposure of the TB domain in the degenerative brain diseases where tauopathy is observed can be usefully utilized in diagnosing or/and treating the degenerative brain diseases.

### DESCRIPTION OF DRAWINGS

FIG. 1 shows a result of confirming the reverse correlation between SQSTM1/p62 protein expression and tau protein expression by performing a transfection experiment.

FIG. 2 shows a result of confirming the coexistence of SQSTM1/p62 protein and tau protein in neurons by performing immunofluorescence staining.

FIG. 3 shows a result of confirming that the TB domain in the SQSTM1/p62 protein and the tau protein are directly bound to each other by performing an immunoprecipitation reaction.

FIG. 5 shows a result of confirming the TB domain specificity after producing a monoclonal antibody (αTB-p62) that specifically binds to the TB domain in the SQSTM1/p62 protein.

FIG. 6 shows a result of confirming that the TB domain in the SQSTM1/p62 protein and the tau protein directly bind to each other using the αTB-p62 antibody.

### BEST MODE

Hereinafter, the present invention will be described in more detail.

The present invention provides a biomarker composition for diagnosing degenerative brain diseases comprising TB (TRAF-6 binding) domain in SQSTM1/p62 protein.

As used herein, the term “biomarker” is a substance capable of diagnosing tissues or cells of a subject with degenerative brain disease by distinguishing them from tissues or cells of a normal control, and includes organic biomolecules such as proteins, nucleic acids, lipids, glycolipids, and glycoproteins, which show an increase or decrease in tissues or cells of a diseased subject compared to the normal control group.

The TB domain may be represented by the amino acid sequence of SEQ ID NO: 1, and may be represented by the nucleotide sequence of SEQ ID NO: 2.

The TB domain is increased in degenerative brain disease and binds to the tau protein to remove the tau protein, so that degenerative brain disease can be prevented or treated.

The degenerative brain disease may be selected from the group consisting of Alzheimer's disease, frontotemporal dementia, Parkinson's disease, amyotrophic lateral sclerosis, progressive supranuclear palsy, corticobasal degeneration, Pick's disease, mild cognitive impairment and dementia, but it is not limited thereto.

In addition, the present invention provides a kit for diagnosing degenerative brain diseases comprising an agent capable of measuring exposure level of TB (TRAF-6 binding) domain in SQSTM1/p62 protein.

The agent may be selected from the group consisting of primers, probes, antibodies, peptides, and aptamers, but it is not limited thereto.

As used herein, the term “primer” refers to a short nucleic acid sequence which has a short free 3-terminal hydroxyl group can form a base pair with a complementary template and serves as a starting point for template strand copying. Primers can initiate DNA synthesis in the presence of reagents for polymerization (i.e., DNA polymerase or reverse transcriptase) and four different nucleotide triphosphates at an appropriate buffer and temperature. In addition, additional features of primers that do not change the basic properties of the primers serving as an initiation point for DNA synthesis may be incorporated as sense and antisense nucleic acids having a sequence of 7 to 50 nucleotides.

As used herein, the term “probe” refers to a nucleic acid fragment such as RNA or DNA corresponding to a few bases or hundreds of bases that can specifically bind other than mRNA, and is labeled so that the presence or absence of a specific mRNA and the amount of expression can be confirmed. The probe may be manufactured in the form of an oligonucleotide probe, a single strand DNA probe, a double strand DNA probe, an RNA probe, or the like. Selection of an appropriate probe and conditions for hybridization can be appropriately selected according to techniques known in the art.

As used herein, the term “antibody” means a specific immunoglobulin directed against an antigenic site, and any product manufactured according to a conventional method in the art or commercially available may be used. In addition, the antibody includes a polyclonal antibody, a monoclonal antibody, and a fragment capable of binding to an epitope. The antibody refers to a complete form having two full-length light chains and two heavy chains, and also includes special antibodies such as humanized antibodies.

As used herein, the term “peptide” has an advantage of high binding power to a target material, and denaturation does not occur even during thermal/chemical treatment. In addition, since the molecular size is small, it can be attached to other proteins and used as a fusion protein. Specifically, since it can be used by attaching it to a polymer protein chain, it can be used as a diagnostic kit and a drug delivery material.

As used herein, the term “aptamer” is a single-stranded nucleic acid (DNA, RNA or modified nucleic acid) that has a stable tertiary structure by itself and can bind to a target molecule with high affinity and specificity. Aptamers are comparable to single antibodies because of their inherent high affinity (usually pM level) and specificity that they can bind to target molecules, and in particular, there is a high potential as an alternative antibody so that it can be referred to as a “chemical antibody”.

In addition, the kit of the present invention may include an antibody that specifically binds to a marker component, a secondary antibody conjugate to which a label to develop color by reaction with a substrate is conjugated, a color developing substrate solution to react with the label, a washing solution, and an enzyme reaction stop solution, and the like, and may be prepared as a plurality of separate packaging or compartments including the reagent components used.

In addition, the present invention provides a method of providing information necessary for diagnosing degenerative brain diseases comprising: (a) measuring an exposure level of TB (TRAF-6 binding) domain in tertiary structure of SQSTM1/p62 protein in a sample isolated from a patient; (b) comparing the exposure level of the TB domain with a control sample; and (c) determining as degenerative brain disease when the exposure level of the TB domain is lower than that of the control sample.

In addition, the present invention provides a method of screening a therapeutic agent for degenerative brain diseases comprising: (a) treating a candidate drug to a sample isolated from a patient; (b) measuring an exposure level of TB (TRAF-6 binding) domain in tertiary structure of SQSTM1/p62 protein before and after treatment with the candidate drug; and (c) selecting a candidate drug that increases the exposure level of the TB domain compared to a control sample.

As used herein, the term “sample isolated from a patient” may include samples such as tissues, cells, whole blood, serum, plasma, saliva, sputum, cerebrospinal fluid, or urine that differ from the control in the exposure level of the TB domain, but it is not limited thereto.

Hereinafter, the present invention will be described in more detail through examples. These examples are only intended to illustrate the present invention in more detail, and it will be apparent to those skilled in the art that the scope of the present invention is not limited by these examples according to the gist of the present invention. The examples of the present invention are provided to more completely explain the present invention to those of ordinary skill in the art.

### EXAMPLE 1

### Analysis of Reverse Correlation between SQSTM1/p62 Protein and Tau Protein using Transfection

The Chinese hamster ovary (CHO) cell line was cultured in DMEM medium supplemented with 10% fetal bovine serum (FBS) and 1% penicillin/streptomycin. Cells were inoculated into a 12-well plate at a density of 1.2×10⁵ cells/well, incubated for 24 hours, and then GFP tagged Tau plasmid constructs (GFP-3R, GFP-4R) and HA tagged p62 plasmid constructs with different concentrations (+, ++, +++) were transfected together using Lipofectamine 2000 (Invitrogen). After 24 hours, cells were recovered, protein was extracted, and Western blot was performed with the same amount of protein. Antibodies used were antibodies against total tau protein (anti-Tau5 antibody), antibodies against phosphorylated tau (anti-pT212 antibody, anti-pT231 antibody, anti-pS199 antibody, depending on the phosphorylation site), and an antibody against actin (anti-actin antibody).

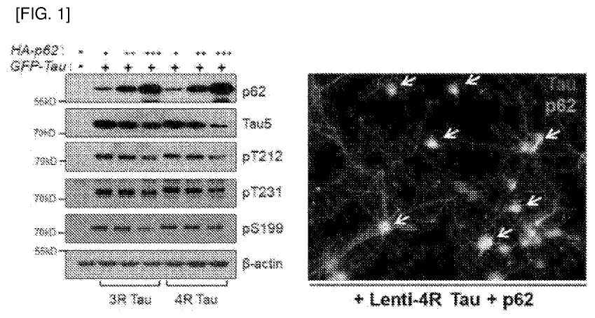

As a result, as shown in FIG. 1, it was confirmed that as the expression of SQSTM1/p62 protein increased in the CHO cell line, the expression of total tau protein and phosphorylated tau protein was reversely decreased (left).

### EXAMPLE 2

### Analysis of Coexistence of SOSTM1/p62 Protein and Tau Protein in Nerve Cells

On the 18th day of pregnancy of Sprague-Dawley rats, fetal cerebral cortex and hippocampal cells were isolated, and then inoculated into a 12-well plate containing coverslips coated with nerve cells at a density of 1.2×10⁵ cells/well, and incubated for 7 days in a 37° C., 5% CO₂ incubator using Neurobasal medium supplemented with B-27 and L-GlutMAX-1. Every 3-4 days, half of the culture medium was replaced with a new medium.

After 7 days of culture, 1 ×10⁶ TU of a lentiviral vector was transduced into cultured primary neurons and cultured for 48 hours, followed by immunocytochemistry. Briefly, the coverslip in which the nerve cells were cultured was taken out and washed twice with cold phosphate buffered saline (PBS), and the cells were fixed at 4° C. for 10 minutes using 4% formaldehyde. Thereafter, the coverslip was washed with PBS, and then immersed in PBS containing 0.1% Triton X-100 and 2% FBS, and the cell membrane was permeated for 5 minutes at room temperature. Thereafter, the cells were reacted with the primary antibody (4R Tau and p62 antibody, 1:1000 dilution) overnight at 4° C., washed three times with PBS, and then reacted with Alexa Fluor 647 (4R Tau) or Alexa Fluor 405 (p62) secondary antibody (all 1: 1000 dilution) at room temperature for 1 hour. Stained cells were observed using a confocal microscope.

As a result, as shown in FIG. 1, it was confirmed that SQSTM1/p62 protein and tau protein were present at the same location in nerve cells in immunofluorescence staining (arrow in the right figure).

### EXAMPLE 3

### Analysis of Direct Binding between TB Domain and Tau Protein in SCISTM1/p62 Protein

3-1. Western Blot

A mutant construct in which Phox and Bem1 (PB1) domains, TRAF6-binding (TB) domains, and Ubiquitin-binding (UBA) domains were each selectively removed using the p62 plasmid structure (Addgene, Cambridge, MA, USA) was produced.

After transfecting the p62 wild type (WT) construct and its mutant construct into the CHO cell line as a vector control or Tau plasmid construct, incubating for 24 hours, and then cells were recovered and Western blot was performed with the same amount of the protein. The results were quantitatively analyzed for each condition using the Image J program, and then determined based on the amount of Tau protein when the Tau plasmid structure was expressed with the vector (100%), and the amount of tau protein was analyzed when expressed with p62 wild-type (WT) or p62 mutant construct (ΔTB) from which the TB domain was removed.

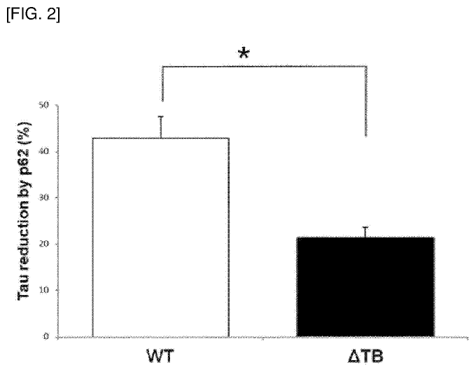

As a result, as shown in FIG. 2, it was confirmed that when the TB domain of the SQSTM1/p62 protein was removed, the effect of reducing tau protein by the SQSTM1/p62 protein was weakened.

3-2. Immunoprecipitation

The mutant constructs from which the p62 wild-type construct and its main domain have been removed (p62Δ_PB1, p62Δ_TB, p62Δ_LIR, p62Δ_UBA) were transfected into the CHO cell line as the tau plasmid construct and cultured for 24 hours. Thereafter, the cells were washed with cold PBS, and for immunoprecipitation, the protein extract of the cells was extracted with NP-40 extraction buffer, and then 1 g of the tau antibody was reacted overnight at 4° C. Then, the immune precipitate was reacted with protein G-agarose bead (30 μL) at 4° C. for 4 hours. Thereafter, the obtained immune precipitate was subjected to electrophoresis on an SDS-PAGE gel to separate proteins, transferred to a nitrocellulose membrane, and reacted with an anti-p62 antibody to perform Western blot.

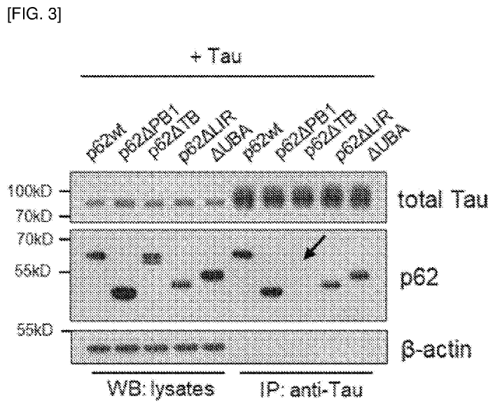

As shown in FIG. 3, as a result of selectively removing the main domain of the SQSTM1/p62 protein, it was confirmed that when the TB domain was removed, the binding of the SQSTM1/p62 protein and the tau protein disappeared (arrow). This shows that SQSTM1/p62 protein and tau protein directly bind through the TB domain.

The amino acid sequence and nucleotide sequence of the TB domain in the SQSTM1/p62 protein are shown in Table 1 below.

TABLE 1

| Amino acid sequence of TB domain | |
|---|---|
| (SEQ ID NO: 1): | |
| If the size of SQSTM1/p62 protein | |
| is 440 aa, the length of 26 aa | |
| corresponding to sequence of | Nucleotide sequence of |
| 225-250 aa | TB domain (SEQ ID NO: 2) |
| SGPSEDPSVNFLKNVGESVAAALSPL | TCTGGTCCATCGGAGGATCCGAGTGT |
| (= Ser Gly Pro Ser Glu Asp Pro Ser | GAATTTCCTGAAGAACGTTGGGGAGA |
| Val Asn Phe Leu Lys Asn Val Gly | GTGTGGCAGCTGCCCTTAGCCCTCTG |
| Glu Ser Val Ala Ala | |
| Ala Leu Ser Pro Leu) | |

### EXAMPLE 4

### Preparation of Antibody Specifically Binding to TB Domain in SCISTM1/p62 Protein

4-1. Antibody construction and analysis of specific binding with TB domain

A monoclonal antibody (αTB-p62) specifically binding to the TB domain in the SQSTM1/p62 protein was prepared by targeting 15 amino acids (N′-SGPSEDPSVNFLKNC-C′) in the TB domain. Briefly, a peptide for the target sequence was prepared, followed by MonoExpress Bronze Antibody Production and antibody purification.

In order to analyze the TB domain specificity of the produced antibody, the p62 wild-type construct and its mutant construct (p62Δ_PB1, p62Δ_TB, p62Δ_LIR/KIR, p62Δ_UBA) were transfected in CHO cell line as the Tau plasmid construct and cultured for 24 hours. Thereafter, the cells were recovered and Western blot was performed with the same amount of protein.

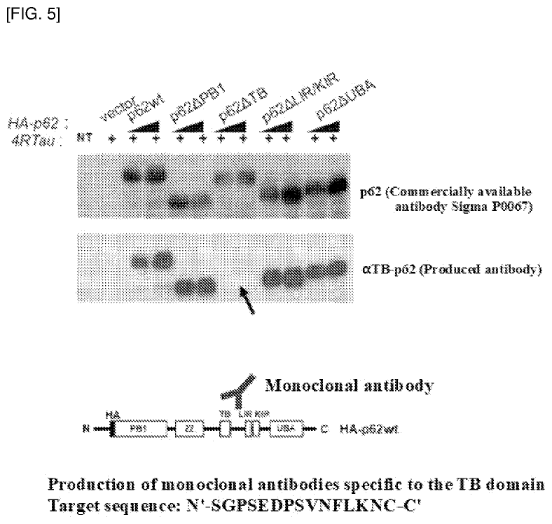

As shown in FIG. 5, as a result of selectively removing the main domain of the SQSTM1/p62 protein, it was confirmed that the reaction of the αTB-p62 antibody disappeared when the TB domain was removed (arrow), so the produced antibody specifically binds to the TB domain.

4-2. Tau protein of TB domain specific antibody (αTB-p62 antibody)

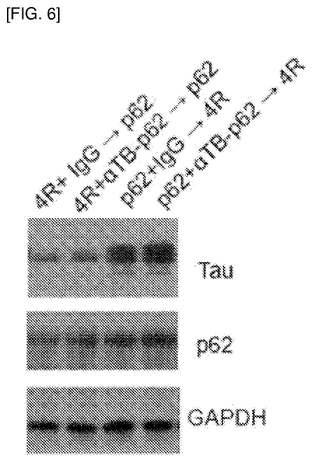

When the p62 wild-type construct and tau protein were simultaneously expressed in the CHO cell line, the concentration of tau protein decreased. As shown in FIG. 6, it was confirmed that the effect of reducing tau protein by p62 was canceled when the αTB-p62 antibody was treated. It was confirmed that the concentration of tau protein was higher in the 2nd and 4th lanes treated with αTB-p62 antibody compared to the 1st and 3rd lanes treated with non-specific IgG. This shows that the αTB-p62 antibody binds to the TB domain of the p62 wild type, thereby inhibiting the binding of the p62-tau protein to inhibit the effect of reducing tau protein by p62.

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

The scope of the present invention is indicated by the claims to be described later, and all changes or modified forms derived from the meaning and scope of the claims and their equivalent concepts should be construed as being included in the scope of the present invention.

1. A biomarker composition for diagnosing degenerative brain diseases comprising TB (TRAF-6 binding) domain in SQSTM1/p62 protein.

2. The biomarker composition for diagnosing degenerative brain diseases of claim 1, wherein the TB domain is represented by amino acid sequence of SEQ ID NO: 1.

3. The biomarker composition for diagnosing degenerative brain diseases of claim 1, wherein the TB domain is represented by nucleotide sequence of SEQ ID NO: 2.

4. The biomarker composition for diagnosing degenerative brain diseases of claim 1, wherein the TB domain binds to a tau protein.

5. The biomarker composition for diagnosing degenerative brain diseases of claim 1, wherein the degenerative brain disease is selected from the group consisting of Alzheimer's disease, frontotemporal dementia, Parkinson's disease, amyotrophic lateral sclerosis, progressive supranuclear palsy, corticobasal degeneration, Pick's disease, mild cognitive impairment and dementia.

6. A kit for diagnosing degenerative brain diseases comprising an agent capable of measuring exposure level of TB (TRAF-6 binding) domain in SQSTM1/p62 protein.

7. The kit for diagnosing degenerative brain diseases of claim 6, wherein the agent is selected from the group consisting of primers, probes, antibodies, peptides and aptamers.

8. A method of providing information necessary for diagnosing degenerative brain diseases comprising:
(a) measuring an exposure level of TB (TRAF-6 binding) domain in tertiary structure of SQSTM1/p62 protein in a sample isolated from a patient;
(b) comparing the exposure level of the TB domain with a control sample; and
(c) determining as degenerative brain disease when the exposure level of the TB domain is lower than that of the control sample.

9. A method of screening a therapeutic agent for degenerative brain diseases comprising:
(a) treating a candidate drug to a sample isolated from a patient;
(b) measuring an exposure level of TB (TRAF-6 binding) domain in tertiary structure of SQSTM1/p62 protein before and after treatment with the candidate drug; and
(c) selecting a candidate drug that increases the exposure level of the TB domain compared to a control sample.


SEQUENCE LISTING (2 sequences)

SEQ ID NO: 1 - protein, 26 aa, Homo sapiens
SGPSEDPSVNFLKNVGESVAAALSPL

SEQ ID NO: 2 - DNA, 78 bp, Homo sapiens
tctggtccatcggaggatccgagtgtgaatttcctgaagaacgttggggagagtgtggcagctgcccttagccctctg